(19) United States
(12) Patent Application Publication
(10) Pub. No.: US 2026/0088156 A1
(43) Pub. Date: Mar. 26, 2026

(54) TECHNIQUE FOR MEDICAL IMAGING CONTROL BASED ON A REQUEST MESSAGE

(71) Applicant: Siemens Healthineers AG, Forchheim (DE)

(72) Inventors: Christian TIETJEN, Fuerth (DE); Sven KOHLE, Erlangen (DE); Gerhard KOHL, Neunkirchen am Brand (DE); Steffen WEICHERT, Wohltorf (DE); Christoph BRACHMANN, Achim (DE); Diego BARRIOS ROMERO, Osterholz-Scharmbeck (DE); Johannes HAUBOLD, Essen (DE)

(73) Assignee: Siemens Healthineers AG, Forchheim (DE)

(21) Appl. No.: 19/339,387

(22) Filed: Sep. 25, 2025

(30) Foreign Application Priority Data
Sep. 26, 2024 (DE) 10 2024 209 357.2

(51) Int. Cl. G16H 30/40; G16H 10/60; G16H 15/00; G16H 50/20

(52) U.S. Cl. CPC G16H 30/40; G16H 10/60; G16H 15/00; G16H 50/20

(57) ABSTRACT

One or more example embodiments relates to a technique for outputting a control signal for at least one of preparing, executing or processing medical imaging of a patient based on a request in natural language. A computer-implemented method comprises receiving a digital request message comprising the request in connection with the medical imaging, wherein the request is formulated in natural language; processing the request, wherein the processing comprises applying at least one first large language model (LLM); and outputting the control signal to an imaging function facility for the at least one of the preparing the medical imaging, the executing the medical imaging or the processing the medical imaging, wherein the output control signal is based on a result of the processing of the request.

### CROSS-REFERENCE TO RELATED APPLICATION(S)

The present application claims priority under 35 U.S.C. § 119 to German Patent Application No. 10 2024 209 357.2, filed Sep. 26, 2024, the entire contents of which are incorporated herein by reference.

### FIELD

A technique is provided for outputting a control signal for preparing, executing and/or processing a result of medical imaging based on a request in natural language. The technique in particular comprises a method, a control unit, a system, a computer program product and a computer-readable storage medium.

### RELATED ART

Radiologists and other clinicians often receive requests in unstructured form, for example in the form of a letter from a referring physician, a question in a chat from a colleague about a specific case, or instructions to prepare a list of patients for a case conference.

Conventionally, radiologists have to work through the unstructured text and view the relevant case(s) in the designated IT systems (for example, RIS, PACS and AV) to obtain an overview. For this purpose, physicians conventionally have to identify patients themselves or manually and, if necessary, import them beforehand, add the patient to a worklist if necessary or create such a worklist, initialize the system accordingly so that it opens the medical record in the correct environment, display the described pathologies in the unstructured documents in the system (for example, select the correct series and jump to the correct layer), search for further documents that confirm the pathologies (for example, laboratory findings or surgical reports) and write up the findings or response. The conventional sequence of many work steps to be performed manually by the physician is time-consuming, prone to human error and limits the achievable image quality and accuracy of an interpretation.

### SUMMARY

One or more example embodiments provides a solution for technical support in the preparation, execution and/or processing of requested medical imaging. Alternatively or additionally, reduces the time required, improves image quality and enables more accurate interpretation.

This is achieved by a method for outputting a control signal for preparing, executing and/or processing a result of medical imaging based on a request in natural language by a control unit, by a system comprising the control unit, by a computer program and/or computer program product and by a storage medium as claimed in the appended independent claims. Advantageous aspects, features and embodiments are described in the dependent claims and in the following description together with advantages.

### BRIEF DESCRIPTION OF THE DRAWINGS

FIG. 1 is a flow diagram of a method for outputting a control signal for preparing, executing and/or processing (for example a result of) medical imaging based on a request in natural language according to a preferred embodiment of the present invention; and

FIG. 2 is an overview of the structure and layout of a control unit for outputting a control signal for preparing, executing and/or processing (for example a result of) medical imaging based on a request in natural language according to a preferred embodiment of the present invention.

### DETAILED DESCRIPTION

The following describes at least one solution with regard to the claimed method for outputting a control signal for preparing, executing and/or processing a result of medical imaging based on a request in natural language and with regard to the claimed control unit. Herein, features, advantages or alternative embodiments can be assigned to the other claimed subject matter (for example, the system, the computer program or a computer program product) and vice versa. In other words: the claims for the control unit and the system comprising the control unit can be improved by features described or claimed in relation to the method. In this case, the functional features of the method are embodied by structural units of the control unit or the system and vice versa.

According to one method aspect, a method (in particular a computer-implemented method) is provided for outputting a control signal for preparing, executing and/or processing (in particular a result of) medical imaging based on a request in natural language. The method comprises a step of receiving a digital request message. The request message comprises a request in connection with medical imaging relating to a patient. The request is formulated in natural language. The method furthermore comprises a step of processing the request comprised in the received digital request message. Processing comprises applying at least one (in particular a first) large language model (LLM). The method furthermore comprises a step of outputting a control signal to an imaging function facility (also referred to as an imaging device) (for example a scanner and/or a configuration device connected to a scanner) for preparing medical imaging, executing medical imaging and/or processing a result of medical imaging. The output control signal is based on a result of processing the request. The output control signal can be intended to control the imaging function facility.

The technique according to one or more example embodiments can significantly improve the preparation (also: planning), execution (also: performance) and/or evaluation of medical images (also: scans). Not only can time be saved, human errors or limitations of possible combinations (for example of scan parameters) can be minimized, image quality can be improved, and/or interpretation can be optimized, for example in that supportive computer tools select suitable data visualizations and/or visualize possible anomalies via image processing.

The method can be executed by a control unit. The control unit can be assigned to a facility that, for example, operates a fleet of scanners (also: medical imaging devices). For example, the control unit can be assigned to a medical facility such as a hospital (also: clinic), a radiology department and/or a radiology practice.

In one exemplary embodiment, the control unit can be localized, for example on a central computer and/or in an imaging system, for example on (or connected to) a scanner. In a further exemplary embodiment, the control unit can be distributed and/or cloud-based, for example in a private cloud of the medical facility.

The request in connection with medical imaging (in short: the request) can be comprised in a request message. The request message can be issued in different data formats, for example as a voice message, text message and/or image message and/or in mixed forms. The request and/or the request message can be converted by suitable digital processing steps into input variables that are suitable for processing in the LLM, such as, for example, into a matrix format and/or tensor format and/or vector representation. The request can comprise a referral (for example from a general practitioner and/or a physician from a specialty other than radiology). Alternatively or additionally, the request can comprise a (for example partial) interpretation request, a medical question and/or request for a diagnosis. The request in connection with medical imaging can be a request to perform or prepare (future) medical imaging. Alternatively, the request can also be a request to answer a medical question in relation to or in connection with medical imaging that has already been performed, such as, for example, producing (partial) findings based on imaging or answering a specific question in relation to imaging. Alternatively or additionally, the request can also be a request to prepare (future) medical imaging.

The digital request message can comprise the request (at least partially) in written form. The request message can be in an unstructured format (for example formulated as free text). The request message can be transmitted via suitable data transmission networks, for example LAN, WLAN, an http(s)-based protocol, radio, etc. Transmission software, for example an email program or a chatbot, for example a messenger app, can be used for this purpose.

Alternatively or additionally, the digital request message can comprise a recorded voice message and/or a recorded physician-patient conversation (for example, a voice mail, a voice message sent via a messenger app and/or a recording of a physician-patient conversation transmitted via HL7).

The method can use more than one LLM (large language model). For example, patient data (for example from a medical record) and/or a medical history can be analyzed based on the request, in particular via a second LLM. Alternatively or additionally, a further (for example third) LLM can be tailored or trained for one or more medical ontologies. Alternatively or additionally, yet a further (for example fourth) LLM can be instructed and/or trained for a target system to be controlled and/or configured (and/or the imaging function facility, for example the scanner that is to execute the medical imaging), for example in order to know the target system (and/or the imaging function facility), for example on the basis of technical documentation.

The first LLM can be used to extract the content relevant for the request in connection with medical imaging from the natural language. For example, the patient's name, a medical imaging modality, a body region and/or a medical question (for example with regard to a suspected diagnosis or clarification of a preliminary interpretation, for example based on a previous physical examination, analysis of symptoms and/or laboratory values) can be extracted from the request via the first LLM, in particular in order to plan medical imaging.

The first LLM can, for example, convert the extracted medical question into a standard format and/or a standard language (for example according to a medical ontology).

The imaging function facility can be a medical imaging device (also: scanner) or functionally interact therewith. The imaging function facility can also be a facility that interacts with the scanner. The imaging function facility can comprise the scanner and/or a configuration device that functionally interacts with the scanner. For example, the imaging function facility can be a facility or a device on which software is implemented that is embodied to execute preparatory steps and/or processes for executing an examination, such as, for example, loading a data set from a database or memory that is assigned to the request message or mechatronic settings for preparing imaging for the patient (for example adjusting the table, initiating measurements, etc.).

Alternatively or additionally, the imaging function facility can be a facility that exchanges data with a radiology information system (RIS) and/or a hospital information system (HIS) or is comprised in an RIS or HIS.

In particular in connection with an RIS, the preparation of the medical imaging can comprise allocating a patient to a scanner with required properties and/or allocating a scan protocol. Alternatively or additionally, in particular in connection with the RIS, the preparation of the medical imaging can comprise prioritizing the patient within a list of patients. Further alternatively or additionally, the preparation (and/or the execution) of the medical imaging on the scanner can comprise adapting a scan protocol.

In particular in connection with the RIS, processing the result of medical imaging can comprise allocating the study (or the recording) created on the scanner to a radiologist and/or performing (for example further) processing jobs (and/or post-processing jobs). Alternatively or additionally, in particular in connection with a PACS, processing the result of medical imaging can comprise allocating a hanging protocol. The result of medical imaging can be a digital two-dimensional (2D) and/or three-dimensional (3D) recording (in short also: measurement or measurement result, and/or image) and/or a (for example 2D or 3D) digital image data set.

The control signal can be a digital data set which is, for example, intended to actuate at least one partial function of the scanner. The control signal can comprise a plurality of signal sections. The signal sections can, for example, be used to encode different control parameters and/or sections of a scan protocol.

The control signal can be sent to the scanner for control or preparatory settings. Alternatively or cumulatively, the control signal can be sent to the configuration device that interacts functionally with the scanner. The configuration device can, for example, be a device intended to configure the scanner, such as, for example, a computer-aided workstation for making certain settings on the scanner or on units connected thereto (for example a patient table). Alternatively or additionally, the configuration device can be part of an (in particular smart and/or CT) scanner system in order, for example, to obtain, collect or evaluate information on motion artifacts and/or metal in the body (in particular to improve the image quality of a scan). Alternatively or cumulatively, the configuration device can be a device that defines a sequence of work steps (for example in the form of a worklist) or the planning of scanner usage (for example as a scheduling device). Further alternatively or additionally, the configuration device can be intended for the administration of a contrast agent (for example specifying a field of view of the scanner) and/or (for example setting and/providing) the dose (for example of radiation and/or contrast agent).

The request in the digital request message can be at least partially formulated in text form. The application of the (in particular first) LLM can comprise processing the request in text form.

In one exemplary embodiment, the request can be typed as a message and/or converted into text form via speech recognition prior to sending.

Speech recognition can be used to convert (also: transcribe) a recorded voice message (for example from a referring physician, a radiologist and/or a medical technical assistant, MTA, in particular a medical technical radiology assistant, MTRA) into text form. Alternatively or additionally, speech recognition (for example using a speech processing program, such as Nuance Dragon Ambient experience, DAX) can be used to transcribe a physician-patient conversation and/or a conversation between medical staff.

In text form, the digital request message can be particularly resource-efficient (for example require little storage space and/or few data transmission resources).

The request can be at least partially comprised in language form (in particular in spoken language form) in the digital request message. The method can comprise a step of transcribing the at least partial language form (in particular spoken language form) of the request into text form. The application of the (in particular first) LLM can comprise processing the request in transcribed text form.

The request can, for example, comprise a voice message (for example from a referring physician) and/or a recorded physician-patient conversation (and/or a recorded conversation between two or more members of the medical staff, for example two physicians).

Combinations of text form and spoken language form can, for example, comprise receiving a patient's name (and optionally an imaging modality) in text form, wherein the medical question was dictated by the physician or was recorded as a voice message.

Transcribing the spoken language form on the recipient side (and/or in the medical facility, for example in the radiological department where medical imaging is to be executed) can have the advantage that voice recognition software can in particular be trained to recognize medical terminology and/or expressions typical of medical-imaging-related requests.

Receiving the digital request message can comprise receiving via an email program and/or via a function for storing and/or processing voice messages (in particular an answering machine function). Alternatively or additionally, receiving the digital request message can comprise receiving via short message service (SMS) and/or via multimedia messaging service (MMS). Further alternatively or additionally, receiving the digital request message can comprise receiving via an instant messaging service (for example via WhatsApp, Signal, Threema, Session and/or Telegram) and/or via an (in particular digital) video conferencing platform and/or connection platform (for example via Teams, Zoom, Skype, Jitsi and/or BigBlueButton). Further alternatively or additionally, receiving the digital request message can comprise receiving via a transmission tool based in particular on a communication standard in the healthcare sector (for example HL7).

The digital request message can be received via a request interface. The request interface can, for example, combine a plurality of information channels, such as, for example, email and chat programs. This allows, for example, a referring physician to be provided with the most convenient option for submitting the request.

Health Level 7 (HL7) is a set of international standards for the electronic exchange of data, in particular medical data, between information systems (for example a medical facility, a central database for digital medical records and/or a health insurance company health insurance provider) in the healthcare sector. The standards are defined by the international organization of the same name.

Processing can comprise a step of selecting an imaging function facility for preparing medical imaging, executing medical imaging and/or processing the result of medical imaging that has already been executed. In particular, processing can comprise a step of selecting a scanner for executing medical imaging. The scanner can be selected from a scanner fleet based on the request.

Alternatively or additionally, processing can comprise a step of requesting a patient's medical history from a digital database and an optional step of processing the requested medical history via a second LLM. Processing the request in connection with medical imaging can be based on the processed medical history.

Alternatively or additionally, processing can comprise a step of querying medical ontology with regard to a medical question assigned to the request in connection with medical imaging. Querying the medical ontology can comprise using a third LLM.

Alternatively or additionally, processing can comprise a step of reserving a time window for medical imaging.

Alternatively or additionally, processing can comprise a step of determining at least one scan parameter and/or determining a scan protocol. The at least one scan parameter and/or the scan protocol can be comprised in the control signal.

The digital database requested for the medical history can comprise a database with electronic health records (EHR) and/or electronic medical records (EMR). Alternatively or additionally, the digital database can be stored in an RIS and/or HIS. Alternatively or additionally, the digital database can comprise a picture archiving and communication system, PACS), RIS and/or HIS.

The medical history can in particular contain information relevant to medical imaging, such as the existence of implants (for example a pacemaker, a hip implant, one or more stents and/or one or more dental implants). Alternatively or additionally, the medical history can comprise a clinical picture that affects the selection of medical imaging, such as, for example, intolerance of and/or allergy to contrast agents, heart disease, claustrophobia (relevant for example for recordings in a tube surrounding the patient) and or difficulty lying still for long periods of time. Further alternatively or additionally, the medical history can comprise an indication of refusal to consent to radiotherapy and/or chemotherapy.

The medical ontology (for example SNOMED, Thieme eRef and/or Radiopaedia) can comprise a set of medical terms (for example relating to symptoms, diagnoses and/or clinical pictures) with relationships or links between them. In some exemplary embodiments, the medical ontology (in short also: the ontology) can be assigned to a specialty and/or an organ. For example, a medical ontology can be constructed for heart disease.

The medical ontology can be queried using a third LLM.

A further (for example fourth) LLM can be intended to understand and/or link the capabilities of the target system (and/or the imaging function facility, for example the scanner).

Selecting the scanner can comprise selecting a modality (for example CT, MRI and/or refinements such as single-photon CT). Alternatively or additionally, selecting the scanner can comprise selecting based on the technical equipment of the scanner (for example the presence of measuring coils, the strength of a basic magnetic field, a permissible patient weight for a patient table and/or a diameter of a tube into which the patient has to be introduced), based on executable scan protocols and/or based on setting options for scan parameters.

Selecting the scanner, determining the at least one scan parameter and/or determining the scan protocols can be based on medical guidelines and/or legal requirements (for example standard operating procedures, SOPs).

Selecting the scanner, determining the at least one scan parameter and/or determining the scan protocols can be rule-based (for example based on a table stored in a data memory). Alternatively or additionally, the scanner, the at least one scan parameter and/or the scan protocol can be selected or determined with the aid of artificial intelligence (AI) and/or a further LLM. For example, the AI can comprise machine learning (ML) based on historical medical images (also: scans). The AI can continue to learn from further scans (for example continuously).

Reserving the time window for the scan (also: “scheduling”) can be based on the existence or avoidance of idle times for the scanner and/or the presence of (in particular medical) operating staff for the scanner. Alternatively or additionally, reserving the time window can take into account the urgency and/or priority (for example after an accident or in the case of a suspected acute anomaly) of the medical imaging in accordance with the request.

Processing can be at least partially agent-based.

An agent can be embodied to execute software and/or a function. A plurality of agents can be provided for different processes and/or interact; these can in particular be implemented on different physical instances (for example, hardware and/or virtualization). For example, a distributed agent architecture can be provided. Alternatively, it is possible to deploy all or some of the agents on a common physical instance.

For example, the processing of the request and/or the application of the first LLM can be executed via a first agent (also: intermediary).

A second agent can be embodied to request (and preferably also process) the medical history via the second LLM.

A third agent can be embodied to query the medical ontology regarding the medical question via a third LLM.

A fourth agent can be embodied to select the scanner. The fourth or a further agent can be embodied to determine the at least one scan parameter and/or the scan protocol.

A fifth agent can be embodied to reserve the time window for the scan.

The agents can be numbered as desired. For example, in one exemplary embodiment, the second agent for requesting and/or processing the medical history may not be provided (and/or may not be activated).

The agents can interact with one another and/or coordinate with one another. For example, the fourth agent can determine the scanner based on the medical history processed (also: analyzed) by the second agent. Alternatively or additionally, for example in the case of high urgency (for example after an accident or in the case of acute illness), the fifth agent and the fourth agent can interact and, for example, reschedule a time window already planned for a less time-critical patient for the urgent patient.

The method can comprise a step of receiving a digital image data set from the scanner. The digital image data set can be based on the output control signal.

The method can alternatively or additionally comprise a step of commissioning the processing of a digital image data set. Processing the digital image data set can comprise classifying (in particular object classification and/or tissue classification). Processing the digital image data set can alternatively or additionally comprise semantic segmentation, determining anatomical landmarks, providing data visualization and/or drafting medical findings.

The method can alternatively or additionally comprise a step of receiving a result of image processing in accordance with the commission.

The method can alternatively or additionally comprise a step of sending medical findings and/or a report in response to the received request. Optionally the report can comprise the received digital image data set.

The digital image data set (also: scan) can correspond to the request.

Processing the image data set can comprise computer-implemented image processing concepts, such as classifying (for example per pixel) according to properties (for example tissue type and/or object), semantic segmentation and/or determining anatomical landmarks (for example via Siemens Healthineers ALPHA and/or BodyGPS software) to support diagnosis and/or image interpretations.

The method can comprise steps of pre-processing, processing and/or post-processing. In the context of the invention, each processing step can be referred to as processing. For example, the terms “pre-processing” and “post-processing” may depend on the processing facility (also referred to as a processor) (in particular the imaging function facility). For example, the detection of lung nodules in CT recordings (also: CT images) on the scanner may be referred to as post-processing (in particular because the detection takes place after the recording, also: the acquisition). The detection of lung nodules in CT recordings on the PACS can be referred to as pre-processing (in particular because the recordings are processed before they are viewed by a radiologist).

Data visualization (also: advanced visualization, AV) can comprise selecting a suitable visualization standard, for example a hanging protocol, marking anomalies and/or marking anatomical landmarks. Alternatively or additionally, data visualization can comprise selecting suitable 2D layers from a 3D image data set.

Receiving the result of the image processing can comprise receiving at a user interface (UI), for example a graphical user interface (GUI). For example, a radiological device and/or an interpretation device can be intended to receive the classification, semantic segmentation, specific anatomical landmarks, the data visualization provided and/or the draft medical findings.

Receiving the result of the image processing can comprise a recommendation for further scans, other examinations and/or a referral to a panel of experts (and/or a case conference, for example a tumor board).

A response service can be implemented that causes the medical findings and/or report to be automatically returned as a response message to the request message, to the sending node or a sending address from which the request message was received, for example from a referring physician. The response service's return message can be sent to a sending address of the sending node and/or to an account at which the digital request message was received. Alternatively or additionally, the return message can be sent to predetermined address (for example email address). For example, there may be a list of authorized requesters with response addresses. Further alternatively or additionally, the digital request message can contain a response address.

One (for example any) or each of the processing steps can comprise sending an information message (for example to medical staff such as a physician, in particular a radiologist, and/or an MTA, in particular an MTRA) regarding a processing result to a user interface (UI). Optionally, one (for example any) or each of the processing steps can comprise receiving a rejection signal, a confirmation signal and/or a verification signal via the UI.

For example, an information message can be sent to indicate that a digital request message has been received. In particular, the information message can comprise the request in natural language. If a request is transcribed at least partially in language form (in particular in spoken language form), the information message or a further information message can comprise the transcribed text form.

Upon completion of the processing step of the request, an information message can, for example, be sent with the result. Alternatively or additionally, an information message comprising the output control signal can be sent.

Alternatively or additionally, an information message can be sent when the digital image data set is received by the scanner, when the processing of a (for example the) digital image data set is commissioned, when the result of the image processing is received, and/or when the medical findings and/or report are sent.

The information message can be sent for monitoring the (in particular computer-implemented) method by medical staff, for example a radiologist. In one exemplary embodiment, the absence of a rejection signal (for example within a predetermined period of time from the sending of the information, for example from one working day to the next) can be interpreted as consent (also: confirmation). This can, for example, apply to the selection of the scanner, scheduling and/or determination of scan parameters and/or a scan protocol.

In one exemplary embodiment, receiving draft medical findings can require rejection or verification, optionally with an intermediate step of modifying the draft medical findings by a human (for example a radiologist).

The imaging function facility can comprise a scanner (in particular a medical scanner).

Medical imaging (in particular via the scanner) can comprise a medical imaging modality. The medical imaging modality can comprise computed tomography (CT), magnetic resonance imaging (MRI), radiography, ultrasound, fluoroscopy, positron emission tomography (PET) and/or single photon emission computed tomography (SPECT).

Fluoroscopy can be performed via a scanner comprising a C-arm.

The method can be executed in a local network of a medical facility in which the imaging function facility is integrated. Optionally, the method can be cloud-based and/or executed on an edge device.

The edge device can be a device that links the local network of the medical facility to the Internet and/or an external network.

According to one apparatus aspect, a control unit is provided for outputting a control signal for preparing, executing and/or processing (for example a result of) medical imaging based on a request in natural language. The control unit comprises a request receiving interface. The request receiving interface is embodied to receive a digital request message comprising a request in connection with medical imaging relating to a patient. The request is formulated in natural language. The control unit furthermore comprises a processing module. The processing module is embodied to process the request message comprised in the received digital request. Processing comprises applying at least one (for example first) LLM. The control unit furthermore comprises a control signal output interface. The control signal output interface is embodied to output a control signal to an imaging function facility for preparing medical imaging, executing medical imaging and/or processing a result of medical imaging, wherein the output control signal is based on a result of processing the request.

The control unit can comprise further interfaces and/or modules, for example a transcription module embodied to transcribe a request comprised at least partially in language form (in particular spoken language form) in the digital request message.

The control unit can be implemented by at least one control node. Alternatively or additionally, the control unit can be implemented locally or distributed, for example cloud-based.

The control unit can be embodied to execute one or each of the method steps according to the method aspect. Alternatively or additionally, the control unit can comprise one or each feature disclosed in relation to the method aspect.

According to one system aspect, a system is provided for outputting a control signal for preparing, executing and/or processing (for example a result of) medical imaging based on a request in natural language. The system comprises a control unit according to the apparatus aspect. The system furthermore comprises at least one imaging function facility (for example a scanner) embodied for preparing medical imaging, executing medical imaging and/or processing a result of medical imaging.

According to a further aspect, a computer program product is provided with program elements that cause a control unit to execute the steps of the method for outputting a control signal for preparing, executing and/or processing (for example a result of) medical imaging based on a request in natural language according to the method aspect when the program elements are loaded into a memory of the control unit.

According to a yet further aspect, a computer-readable medium is provided on which program elements are stored which can be read and executed by control unit in order to perform steps of the method for outputting a control signal for preparing, executing and/or processing (for example a result of) medical imaging based on a request in natural language according to the method aspect when the program elements are executed by the control unit.

The above-described properties, features and advantages and the manner in which they are achieved will become clearer and more plainly comprehensible in the light of the following description and the exemplary embodiments explained in more detail in connection with the drawings. The following description does not limit the invention to the embodiments contain therein. The same components or parts may be provided with the same reference symbols in different figures. In general, the figures are not true to scale.

It should be understood that a preferred embodiment of the present invention can also be any combination of the dependent claims or the aforementioned embodiments with the respective independent claim.

Reference symbols in the claims should not be understood as limiting the scope of application.

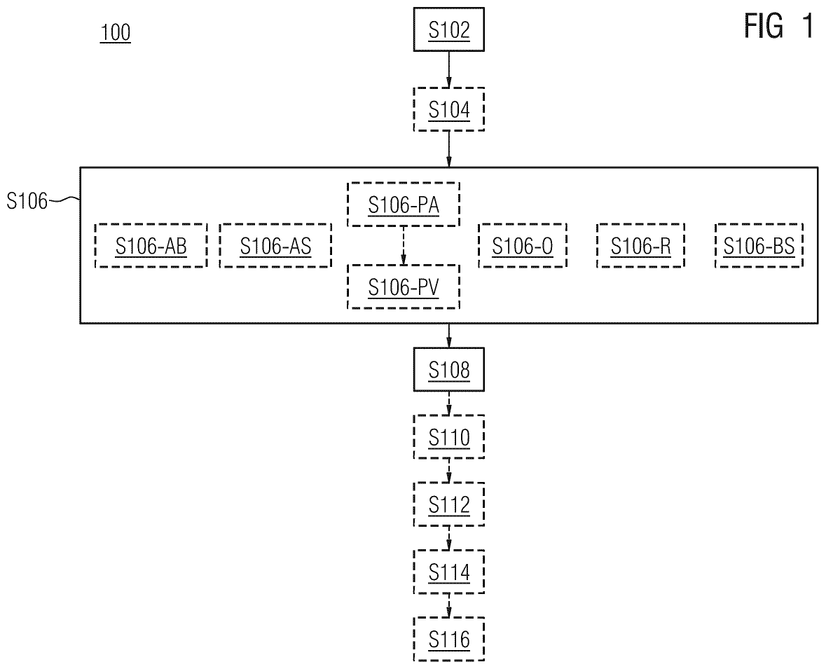

FIG. 1 is a schematic view of an exemplary flowchart of a method 100 (in particular a computer-implemented method) for outputting a control signal for preparing, executing and/or processing (for example a result of) medical imaging based on a request in natural language.

The method 100 comprises a step S102 of receiving a digital request message. The request message comprises a request in connection with medical imaging relating to a patient. The request is formulated in natural language.

The method 100 furthermore comprises a step S106 of processing the request comprised in the digital request message received in S102. Processing S106 comprises applying at least one first large language model (LLM).

The method 100 furthermore comprises a step S108 of outputting a control signal to an imaging function facility (for example to a scanner) for preparing medical imaging, executing medical imaging and/or processing a result of medical imaging. The control signal output in S108 is based on a result of processing S106 the request.

The request can be comprised at least partially in language form (in particular spoken language form) in the digital request message. The method 100 can furthermore comprise a step S104 of transcribing the at least partial language form (in particular spoken language form) of the request into text form. Applying the first LLM can comprise processing the request in the transcribed S104 text form.

During processing S106, the method 100 can comprise a step S106-AB of selecting an imaging function facility for preparing medical imaging and/or processing (in particular a result of) medical imaging. Alternatively or additionally, during processing S106, the method 100 can comprise a step S106-AS of selecting a scanner as an imaging function facility for executing medical imaging. The scanner can be selected S106-AS from a scanner fleet based on the request.

During processing S106, the method 100 can alternatively or additionally comprise a step S106-PA of requesting (and optionally obtaining) a patient's medical history from a digital database and an optional step S106-PV of processing the requested S106-PA medical history via a second LLM. Processing S106 the request in connection with medical imaging can be based on the processed S106-PV medical history.

During processing S106, the method 100 can alternatively or additionally comprise a step S106-O of querying a medical ontology with regard to a medical question assigned to the request (and in particular obtaining a query result from the medical ontology). Querying S106-O the medical ontology can comprise using a third LLM.

During processing S106, the method 100 can alternatively or additionally comprise a step S106-R of reserving a time window for medical imaging.

During processing S106, the method 100 can alternatively or additionally comprise a step S106-BS of determining at least one scan parameter and/or of determining a scan protocol. The at least one scan parameter and/or the scan protocol can be comprised in the control signal.

The method 100 can comprise a step S110 of receiving a digital image data set from the scanner. The digital image data set can be based on the control signal output in S108.

The method 100 can comprise a step S112 of commissioning processing of a digital image data set (for example, the digital image data set received in step S110). Processing the digital image data set can comprise classifying (in particular object classification and/or tissue classification), semantic segmentation, determining anatomical landmarks, providing data visualization and/or drafting medical findings.

The method 100 can comprise a step S114 of receiving a result of image processing (in particular in accordance with the commissioning S112).

The method 100 can comprise a step S116 of sending medical findings and/or a report in response to the request received in S102. Optionally, the report can comprise the digital image data set received in S110.

In addition to each one of the aforementioned processing steps S102; S104; S106; S108; S110; S112; S114; S116, the method 100 can furthermore comprise sending an information message regarding a processing result to a user interface (UI), and optionally receiving a rejection signal, a confirmation signal and/or a verification signal via the UI.

The sequence of method steps is shown in FIG. 1 merely by way of example. In particular, in alternative embodiments, the steps S112; S114; S116 of processing, receiving the processing result and sending the associated medical findings and/or report regarding medical imaging can also be executed prior to the steps S106; S108 of processing the request and outputting the corresponding control signal.

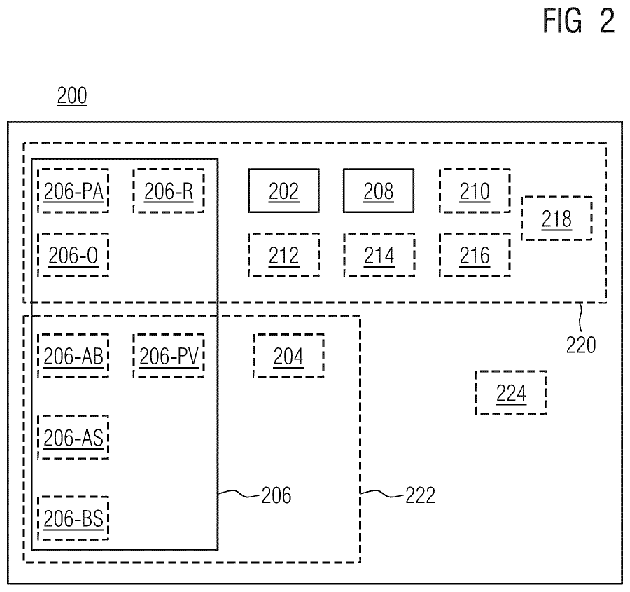

FIG. 2 is a schematic view of an exemplary embodiment of a control unit 200 for outputting a control signal for preparing, executing and/or processing (for example a result of) medical imaging based on a request in natural language.

The control unit 200 comprises a request receiving interface 202 embodied to receive a digital request message comprising a request in connection with medical imaging relating to a patient. The request is formulated in natural language.

The control unit 200 furthermore comprises a processing module 206 embodied to process the request comprised in the received digital request message. Processing comprises applying at least one (for example first) LLM.

The control unit 200 furthermore comprises a control signal output interface 208 embodied to output a control signal to an imaging function facility (for example a scanner) for preparing medical imaging, executing medical imaging and/or processing (in particular a result of) medical imaging. The control signal output is based on a result of processing the request.

The control unit 200 can furthermore comprise a transcription module 204. The request can be comprised at least partially in language form (in particular spoken language form) in the digital request message. The transcription module 204 can be embodied to transcribe the at least partial language form (in particular spoken language form) of the request into text form. Applying the (for example first) LLM can comprise processing the request in transcribed text form.

The control unit 200 can comprise a first module 206-AB embodied to select an imaging function facility for preparing medical imaging and/or processing the result of medical imaging. Alternatively or additionally, the control unit 200 can comprise a second selection module 206-AS embodied to select a scanner as an imaging function facility for executing medical imaging. The scanner can be selected from a scanner fleet based on the request.

The control unit 200 can comprise a request interface 206-PA embodied to request a patient's medical history from a digital database. The control unit 200 can furthermore comprise a processing module 206-PV embodied to process the requested (and in particular obtained) medical history via a (for example further and/or second) LLM. Processing the request in connection with medical imaging can be based on the processed medical history.

The control unit 200 can comprise a query interface 206-O embodied to query a medical ontology with regard to a medical question assigned to the request in connection with medical imaging. Querying the medical ontology can comprise using a (for example further and/or third) LLM.

The control unit 200 can comprise a reservation interface 206-R embodied to reserve a time window for medical imaging.

The control unit 200 can comprise a determination module 206-BS embodied to determine at least one scan parameter and/or a scan protocol. The at least one scan parameter and/or the scan protocol can be comprised in the control signal.

The control unit 200 can comprise a first receiving interface 210 embodied to receive a digital image data set from the scanner. The digital image data set can be based on the output control signal.

The control unit 200 can comprise a commissioning interface 212 embodied to commission processing of a digital image data set. Processing of the digital image data set can comprise classifying (in particular object classification and/or tissue classification), semantic segmentation, determining anatomical landmarks, providing data visualization and/or drafting medical findings.

The control unit 200 can comprise a second receiving interface 214 embodied to receive a result of image processing (in particular in accordance with the commissioning).

The control unit 200 can comprise a sending interface 216 embodied to send medical findings and/or a report in response to the received request. Optionally, the report can comprise the received digital image data set.

Each interface can be embodied as a module. For example, the request interface 206-PA for the medical history can be a medical history module embodied to first request the medical history from a digital database (in particular arranged outside the control unit 200), obtain it (if available there) and process it with regard to the request in connection with medical imaging.

The control unit 200 can comprise a user interface (UI) 218 embodied to provide an information message regarding a processing result to a user (in particular medical staff) and, optionally, to receive a rejection signal, a confirmation signal and/or a verification signal.

The different interfaces 202; 206-PA, 206-O, 206-R; 208; 210; 212; 214; 216; 218 can be implemented by a general input-output interface 220.

The transcription module 204, the first and/or second selection module 206-AB; 206-AS, the processing module 206-PC, the determination module 206-BS and/or each further module can be implemented by a processor 222.

The control unit 200 can furthermore comprise a memory 224. Instructions for executing the method 100 can be stored in the memory 224. Alternatively or additionally, results of steps or intermediate steps of the method 100 can be stored in the memory 224.

The control unit 200 can be embodied to execute the method 100.

The technique according to one or more example embodiments (for example comprising the method 100, the control unit 200 and/or a system comprising the control unit 200) can also be referred to as an interface for initializing and configuring IT systems based on unstructured messages.

The technique according to one or more example embodiments enables processes when receiving requests (also: requests in natural language, also: unstructured form) to be automated by analyzing the unstructured document (also: the request message) by a control unit or a system in order to open the system accordingly and generate a report or response based on the interaction between the radiologist and the system that can be sent back by the radiologist. One or more LLMs are used as the technological basis for this.

According to one exemplary embodiment, the control unit (or the system comprising the control unit) comprises an interface (in particular a request receiving interface) in order to be able to directly receive such unstructured documents (and/or requests in natural language) or requests, for example via an integrated mail function (for example mail@abc-space.krankenhaus-x.de) and/or via an integrated chat function such as Teams, Threema and/or WhatsApp.

In the exemplary embodiment, the unstructured document is forwarded directly to the system.

According to the exemplary embodiment, the system analyzes the content of the unstructured document with the aid of one or more LLMs. The analysis can comprise which patient or which patients are mentioned and/or whether the patient or patients are already included in a database to which the recipient has access. If not, the patient or patients can, for example, be imported from a PACS.

Alternatively or additionally, the analysis can comprise creating a worklist with all patients if a plurality of patients is mentioned, in particular for a particular case conference or tumor board. Alternatively, the patients can be added to an existing worklist with the correct priority in that worklist. Alternatively or additionally, patients can be deleted from a worklist (for example if they have died or moved away).

Alternatively or additionally, the analysis can comprise opening a medical record using the given set of rules (for example selecting a suitable AV application and/or a suitable hanging protocol from an IT system, such as RIS or PACS).

Alternatively or additionally, the analysis can comprise checking whether pre-processing and/or post-processing may need to be initiated in order to be able to identify pathologies described in the document.

Alternatively or additionally, the analysis can comprise checking which anatomical and/or pathological circumstances are described in the findings. The best possible study and/or series can be selected in order to be able to represent the circumstances. For example, algorithms that recognize landmarks in the body or other algorithms can be used in order to be able to jump to the corresponding layers in the image (or images).

Alternatively or additionally, the analysis can comprise opening findings and/or further sources described in the findings, for example findings relating to described laboratory parameters.

Further alternatively or additionally, if differential diagnoses are described, the analysis can comprise searching for further documents that can help to exclude the differential diagnosis. The way in which such differential diagnoses can be excluded can be automatically looked up using one or more LLMs in one or more medical ontologies or databases (such as SNOMED, Thieme eRef or Radiopaedia).

Hence, the unstructured document can be used to preconfigure the system as far as possible.

The “back-office activities” described, such as searching for and loading a patient's medical record (in short also: a patient), and/or visualizing the pathologies conventionally take up a large portion of a radiologist's working time. If these activities are automated in accordance with the technique according to one or more example embodiments, radiologists can work much more efficiently.

An additional advantage of the technique according to one or more example embodiments can be that less investment and IT integration effort is required for the medical facility (for example clinic). A similar convenient integration is conventionally only possible if the medical facility (for example clinic) invests in a comprehensive tumor board management system, which in turn entails costs, effort, time and policy coordination.

According to one or more example embodiments, the technique (for example the system) offers an email function and/or chat function to which documents are sent and the system responds accordingly to the email and/or chat. A response can be composed from the user/system interaction and sent via the email and/or chat function.

Independent of the specific grammatical term usage (for example patient, medical staff, physician, radiologist and/or MTRA), individuals with male, female or other gender identities are included within the term.

Unless already explicitly described, individual embodiments or aspects and features thereof described with reference to the drawings can be combined with one another or exchanged without limiting or extending the scope of the described invention, provided such a combination or a such an exchange is reasonable and in the spirit of the present invention. Advantages described with reference to a specific embodiment of the present invention or with regard to a specific figure are, wherever applicable, also advantages of other embodiments of the present invention.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

1. A computer-implemented method for outputting a control signal for at least one of preparing, executing or processing medical imaging of a patient based on a request in natural language, the method comprising:
receiving a digital request message comprising the request in connection with the medical imaging, wherein the request is formulated in natural language;
processing the request, wherein the processing comprises applying at least one first large language model (LLM); and
outputting the control signal to an imaging function facility for the at least one of the preparing the medical imaging, the executing the medical imaging or the processing the medical imaging, wherein the output control signal is based on a result of the processing of the request.

2. The method of claim 1, wherein the request is at least partially formulated in text form, and wherein the applying the first LLM comprises processing the request in text form.

3. The method of claim 1, wherein the request is comprised at least partially in language form and the method furthermore comprises:
transcribing the at least partial language form of the request into text form, wherein the applying the first LLM comprises processing the request in the transcribed text form.

4. The method of claim 1, wherein the receiving the digital request message comprises receives the digital request message via at least one of,
an email program;
a function for at least one of storing or processing voice messages, in particular an answering machine function;
at least one of short message service (SMS) or multimedia messaging service (MMS);
an instant messaging service;
a video conference platform; or
a transmission tool based on a communication standard.

5. The method of claim 1, wherein the processing the request comprises at least one of:
selecting an imaging function facility for at least one of preparing the medical imaging or processing the result of the medical imaging;
selecting a scanner as an imaging function facility for executing the medical imaging, wherein the scanner is selected from a scanner fleet based on the request;
requesting a medical history of the patient from a digital database, wherein the processing the request is based on the medical history of the patient;
querying a medical ontology with regard to a medical question assigned to the request, wherein the querying the medical ontology comprises using a second LLM;
reserving a time window for the medical imaging; or
determining at least one of at least one scan parameter or one scan protocol, wherein the at least one of the at least one scan parameter or the one scan protocol are comprised in the control signal.

6. The method of claim 1, wherein the processing the request is at least partially agent-based.

7. The method of claim 1, further comprising at least one of:
receiving a digital image data set from a scanner, wherein the digital image data set is based on the output control signal;
commissioning processing of a digital image data set;
receiving a result of image processing in accordance with the commission; or
sending at least one of medical findings or a report in response to the received request, wherein the report optionally comprises the received digital image data set.

8. The method of claim 1, wherein the processing the request further comprises:
sending an information message regarding a processing result to a user interface (UI).

9. The method of claim 1, wherein the imaging function facility comprises a medical scanner.

10. The method of claim 1, wherein the medical imaging comprises a medical imaging modality.

11. The method of claim 1, wherein the method is executed in a local network of a medical facility in which the imaging function facility is integrated.

12. A control unit configured to output a control signal for at least one of preparing, executing or processing medical imaging based on a request in natural language, the control unit comprising:
a request receiving interface configured to receive a digital request message comprising the request in connection with the medical imaging relating to a patient, wherein the request is formulated in natural language;
a processor configured to process the request which includes applying at least one first large language model (LLM); and
a control signal output interface configured to output a control signal to an imaging function facility for the at least one of the preparing medical imaging, the executing medical imaging or the processing a result of the medical imaging, wherein the output control signal is based on a result of processing the request.

13. A control unit configured to perform the method of claim 2.

14. A system comprising:
the control unit of claim 12; and
at least one imaging function facility configured to at least one of prepare the medical imaging, execute the medical imaging or process the result of the medical imaging.

15. A non-transitory computer readable medium with program elements, when executed by a control unit, cause the control unit to perform the method of claim 1.

16. The method of claim 3, wherein the request is comprised at least partially in spoken language form.

17. The method of claim 4, wherein the function for at least one of storing or processing voice messages is an answering machine function.

18. The method of claim 7, wherein the processing the digital image data set comprises at least one of:
classifying, in particular object classification and/or tissue classification;
semantic segmentation;
determining anatomical landmarks;
providing data visualization; or
drafting medical findings.

19. The method of claim 7, wherein the report optionally comprises the received digital image data set.

20. The method of claim 8, wherein the processing the request further comprises:
receiving at least one of a rejection signal, a confirmation signal or a verification signal via the UI.
